(19) United States
(12) Patent Application Publication
(10) Pub. No.: US 2005/0244831 A1
(43) Pub. Date: Nov. 3, 2005

(54) Target dna

(76) Inventor: Gerard Brady, Charlesworth (GB)

Correspondence Address: DAVIS WRIGHT TREMAINE LLP, 865 FIGUEROA STREET, SUITE 2400, LOS ANGELES, CA, 90017-2566, US

(21) Appl. No.: 10/523,318

(22) PCT Filed: Jun. 23, 2003
(86) PCT No.: PCT/GB03/02710

§ 371 Date: Feb. 1, 2005

(30) Foreign Application Priority Data
Aug. 3, 2002 (GB) 0218089.1

(51) Int. Cl. C12Q 1/68; C12P 19/34; C12N 15/85

(52) U.S. Cl. 435/6; 435/455; 435/91.2

(57) ABSTRACT

The invention provides a collection of labelled target DNA molecules which are exonuclease derivatives of double-stranded DNA. Collections of target DNA according to the invention have greater sensitivity than previously described targets. The collections of target DNA molecules are preferably derived from double-stranded cDNA. The invention further provides methods and kits for producing collections of labelled target DNA. Advantageously the collections may be derived from double-stranded cDNA prepared in the presence of acetate buffers.

The present invention relates to collections of labelled target DNA.

Recent years have seen a growth in the realisation of the importance of gene expression in the control of biological activities. It is known that expression of specific subsets of genes regulate tissue formation and organogenesis during development and also the properties of adult tissues. Patterns of gene expression influence not only the structure and composition of specific tissues, but also the tissues' responses to various stimuli. These structures, composition and responses, and the patterns of gene expression encoding them, are distinctive markers for individual tissues.

At a more complex level the pattern of genes expressed by whole organisms may be characteristic of specific individuals and provide an insight into their biological status. For instance, there is growing evidence that the pattern of genes expressed by an individual may influence factors such as the individual's predisposition to particular diseases or their responsiveness to certain therapeutic agents.

The current challenge to biologists is to learn how the products of the around 20-40,000 identified human genes interact to produce the complexity exhibited by higher eukaryotes. To a large extent the biological character of a cell can be inferred from the profile of genes it expresses. Although an examination of mRNA or protein expression patterns alone does not directly address function, the knowledge of when and where a gene is expressed can provide valuable insights as to the potential role of a gene and has historically been instrumental in the discovery of developmentally regulated genes. Recognition of the value of the examination of expression patterns led to the development of a plethora of advanced mRNA profiling technologies such as cDNA microarrays (Duggan et al., 1999), SAGE (Velculescu et al., 1995), and cDNA display (Liang and Pardee, 1992) aimed at the simultaneous measurement of tens to several thousand genes in the target samples. Application of these profiling technologies to clinical diseases, such as cancer has confirmed the utility of profiling and provided useful diagnostic and prognostic assays (Shipp et al., 2002; Staunton et al., 2001; van't Veer et al., 2002).

Despite the success of these approaches at the molecular level by identifying patterns of expression exhibited generally by relatively homogeneous cellular samples the cellular complexity of higher eukaryotes still presents a major obstacle to expression profiling.

Over the last 30 years a variety of molecular techniques have been developed for the analysis of gene-expression. In general methods focussed either on the identification and characterisation of genes (either individual genes or networks of related genes) or the characterisation of the input tissue or cell based on a characteristic profile of expressed genes. Although conventional nucleic acid hybridization techniques (such as northern and dot blots) have been used for many years to analyse a small number of genes and samples there have been a variety of advanced mRNA profiling technologies such as cDNA microarrays (Duggan et al., 1999), SAGE (Velculescu et al., 1995), and cDNA display (Liang and Pardee, 1992) which have been recently developed to allow the simultaneous measurement of tens to several thousand genes in the target samples.

Many of the techniques for analysis of gene-expression described above require the use of labelled target DNA capable of binding to complementary DNA sequences in reference samples. In order to take both full advantage of and to extend recent improvements in gene-expression analysis it is important that the labelled target DNA be sensitive, that is to say having a high binding affinity for complementary DNA sequences. It is also beneficial to be able to produce labelled target DNA from small samples, ideally single cells, since this allows a greater range of cell types to be used (since it obviates a requirement for large numbers of cells), and improves confidence that the starting population is “pure”, rather than representing a mixed population of cell types such as is found in many tissue samples. Furthermore, it is advantageous if labelled target DNA can be produced rapidly, by cheap simple techniques. Unfortunately many known collections of labelled target DNA suffer from disadvantages in that they have relatively low sensitivity, or are prepared by laborious, complicated or expensive techniques.

It is an object of the present invention to obviate or mitigate the disadvantages associated with the prior art.

According to the present invention there is provided a collection of labelled target DNA molecules which are exonuclease derivatives of double-stranded DNA.

Collections of labelled target DNA molecules according to the invention provide a number of advantages over prior art target DNA collections, as set out below. Briefly, target DNA collections of the invention provide advantages in terms of their enhanced sensitivity, their ability to be prepared from small samples, and their ease and cost of preparation.

Collections of labelled target DNA molecules according to the invention have greater sensitivity than previously described targets since the single-stranded target DNA molecules of the invention are not susceptible to “self-hybridisation”. Thus collections of labelled target DNA according to the invention, when used in a hybridisation-based assay, are more readily able to hybridise with complementary DNA sequences in a reference sample, should such sequences be present. Furthermore, preparation of the target molecules is more flexible, cheaper and simpler than prior art techniques. These advantages arise from the fact that the collection of target molecules can be prepared from small amounts of starting material (thereby avoiding costly purification steps and increasing the variety of samples from which labelled target DNA can be prepared), and can be prepared using cheap, simple techniques. Furthermore, since labelled target DNA of the invention can be prepared from samples as small as a single cell, it is possible to ensure that the starting population from which the target DNA is prepared represents a pure population as opposed to a mixture of different cell types.

The collection of labelled target DNA molecules may be prepared by a number of different methods. The methods described below are simple allowing easy, cost-effective preparation of the collections of labelled target DNA.

Advantageously the double-stranded DNA from which the labelled target DNA is derived may be cDNA. Thus the labelled target DNA may be representative of a pattern of gene expression in the sample from which the cDNA is derived. One method suitable for the preparation of a collection of labelled target DNA molecules according to the invention is to subject double-stranded DNA such as cDNA, or a derivative thereof (e.g. a DNA population produced by total or partial amplification of the cDNA population), to exonuclease digestion such that a collection of essentially single-stranded DNA molecules is produced, and then to label these single-stranded molecules.

Conveniently the single-stranded molecules may be labelled by incorporation of labelled nucleotides at the 3′ end of the single-stranded DNA molecules using the template-independent DNA polymerase terminal transferase.

An alternative method by which collections of the invention may be prepared is to treat double-stranded DNA, such as cDNA or a derivative thereof, to obtain a labelled double-stranded DNA population and then to effect exonuclease digestion of the labelled DNA population. Production of labelled double-stranded DNA from the non-labelled double-stranded DNA population (or derivative) may, for example, be effected by addition of labelled nucleotides via the DNA polymerase terminal transferase (as described above).

Alternatively a labelled double-stranded DNA population may be derived from the non-labelled DNA population (such as cDNA or a derivative thereof) through amplification of the original non-labelled DNA by well-known polymerase chain reaction (PCR) techniques using labelled nucleotides. The labelled double-stranded DNA population may then be subjected to exonuclease digest in order to produce a substantially single-stranded labelled DNA population. In cases in which the label is incorporated using PCR this provides an advantage in that the efficiency of label incorporation can be readily assessed by gel electrophoresis and/or real-time quantitative PCR.

Conveniently labelled double-stranded DNA representative of gene expression in a sample of interest may be prepared using primers comprising a homopolymer T tract (for example CATCTCGAGCGGCCGCTTTTTTTTTTTTTTTTTTTTTTTT). An example of this amplification technique is described in Brady et al. (1990). When combined with homopolymer tailing (for example using terminal transferase) PCR using such primers produces a population of DNA molecules, in which all molecules have a poly-T region at one end and a poly-A region at the other. This technique has the advantage that a single oligonucleotide can be used for the initial and all subsequent PCR amplifications. The technique also obviates the need to create new priming sites within the molecules to be amplified, since each molecule produced by amplification contains a poly-A region that can anneal to a poly-T region in the primer allowing further rounds of amplification.

Primers comprising a poly-T tail (as described above) may also comprise a further sequence of nucleotides in addition to the tail region. Such further nucleotides may be selected to allow the incorporation into DNA molecules, produced by PCR using these primers, of regions that may be advantageous for the further amplification or subsequent use of molecules produced. For example primers may be designed such that they will incorporate “anchor” sequences (thereby enabling improved specificity of subsequent PCR) or cloning sites (allowing subsequent manipulation of amplified DNA products). Suitable sequences for incorporation into such primers to achieve these purposes would be immediately appreciated by one skilled in the art.

It will be appreciated that the methods described above may be effected by the use of a kit according to appropriate instructions. Accordingly there is provided a kit for the preparation of a collection of labelled target DNA molecules according to the invention, the kit comprising:
(i) an exonuclease;
(ii) terminal transferase; and
(iii) labelled nucleotides.

There is also provided a kit for the preparation of a collection of labelled target DNA molecules according to the invention, the kit comprising:
(i) an exonuclease;
(ii) primers; and
(iii) labelled nucleotides.

A collection of target DNA molecules according to the invention may be labelled by incorporation of labelled nucleotides within the DNA molecules. Labelled nucleotides may incorporate a detectable moiety, or may contain a functional group (e.g. an amino group) that is subsequently able to react with a detectable moiety. Suitable detectable moieties include fluorescent moieties (flurophores), radio-labelled moieties, and enzymes capable of producing a chromogenic reaction with a suitable substrate. In a preferred embodiment DNA molecules according to the invention are directly labelled by incorporation of nucleotides labelled with fluorescent moieties. This technique provides the advantage that relatively small quantities of fluorescent label are required. This has obvious benefits in terms of reducing the cost associated with the production of labelled target DNA. Suitable examples of commercially available fluorescently labelled nucleotides include FluoroLink nucleotides, which are supplied by Amersham Pharmacia Biotech.

In a preferred embodiment of the invention the non-labelled double-stranded DNA from which the labelled single-stranded DNA population is derived is globally amplified cDNA. By globally amplified cDNA we mean cDNA in which DNA molecules representing gene expression retain the same relative abundance as the mRNA transcripts from which they are derived.

There are a number of known techniques by which globally amplified cDNA suitable for use in the invention may be produced. Most preferably the global amplified cDNA is prepared from mRNA using limiting concentrations of nucleotides and a relatively short incubation time in order to limit cDNA synthesis. This ensures that, no matter what the length of the original mRNA transcript, all cDNA molecules produced are of approximately the same relatively small size. Since all the cDNA molecules are of approximately equal size subsequent amplification of the cDNA results in equal reproduction of all the cDNA molecules present. This ensures that the amplified cDNA produced reflects the original relative abundance of the mRNA present in the biological sample. Suitable protocols for the production of global amplified cDNA of this nature are provided in Brady et al. 1990, Cumano et al. 1992 and Brady et al. 1993. In addition to the advantage of allowing the production of amplified populations of cDNA that maintain the relative abundance of the original mRNA the use of global amplified cDNA also provides other advantages. For example global amplified cDNA can be derived either directly from one or more freshly isolated living cells without the need for RNA isolation, or from mRNA purified from a biological sample. Additionally, the production of global cDNA is well suited to automation, providing advantages in terms of ease and speed of use.

The use of globally amplified cDNA in the production of collections of labelled target DNA according to the invention provides a number of advantages. A first advantage arises from the fact that globally amplified cDNA can be produced from samples as small as a single cell, which may typically contain in the region of 20 pg total RNA. Since conventional techniques for the production of collections of target DNA typically require starting quantities of RNA in the region of 20 μg the ability to work with single cells represents a million fold increase template sensitivity. A second advantage of the use of globally amplified cDNA is that large amounts of DNA can be made, which can be readily and simply checked by methods such as gel electrophoresis and/or real-time quantitative PCR prior to and/or following incorporation of label. This provides advantages not only in terms of ease of production, but also in that it avoids the costs associated with inefficient labelling of target DNA molecules and ineffective use or wastage of arrays.

One method by which global amplified cDNA for use in accordance with the invention may be prepared comprises the following steps:
a) preparing a global cDNA population representative of gene expression in a biological sample of interest from mRNA of the sample by using primers and limiting concentrations of nucleotides;
b) homopolymer tailing the global cDNA population; and
c) amplifying the tailed global cDNA population.

Preferably step a) comprises the reverse transcription of mRNA from the biological sample of interest using primers capable of binding to the poly A tail of the mRNA. As described above, the reverse transcription is preferably carried out in the presence of limiting concentrations of nucleotides in order to limit the length of the transcripts produced.

The global cDNA population produced in step a) is preferably homopolymer such that a population of double-stranded DNA molecules that have both homopolymer A and homopolymer T tracts is produced. Homopolymer tailing may be effected using terminal transferase.

It is preferred that at least one of steps a) and b) is effected in the presence of an acetate buffer. Preferably both steps a) and b) are effected in the presence of an acetate buffer. The use of acetate buffers produces conditions that more closely approximate physiological conditions, and thereby improves the sensitivity and yield of the reaction. A preferred acetate buffer suitable for use in the preparation of cDNA for use according to the invention comprises Tris acetate incorporating potassium acetate and/or magnesium acetate. Preferably the acetate buffer comprises 2-200 mM Tris Acetate, 5-500 mM potassium acetate and 1-10 mM magnesium acetate.

It is also preferred that at least one of steps a) and b) is effected in the presence of bovine serum albumen (BSA). The presence of BSA in the reaction mixture during the reverse transcription and homopolymer tailing steps significantly, and surprisingly, increases the efficiency of the reaction. Preferably the concentration of BSA during step a) is between 60 and 90 μg/ml, more preferably between 70 and 80 μg/ml, and most preferably between 72 and 77 μg/ml. Preferably the concentration of BSA during step b) is between 30 and 45 μg/ml, more preferably between 35 and 40 μg/ml, and most preferably between 36 and 39 μg/ml.

A further preferred modification of the method by which global amplified cDNA may be prepared is to undertake the homopolymer tailing step, step b), in the presence of CoCl₂. It has been found that the presence of CoCl₂ causes a surprising increase in the efficiency of cDNA production and thus significantly increases cDNA yield per unit starting mRNA. Preferably the concentration of CoCl₂ is between 0.5-1.5 mM, more preferably between 0.8-1.2 mM, and most preferably between 0.9-1.1 mM (e.g. 1 mM).

It is preferred that homopolymer tailing, step b), is performed in the absence of dithiothreitol (DTT). Surprisingly, the absence of DTT increases the efficiency, and so yield, of the reaction. This finding is surprising since it has previously been believed that DTT confers an advantage in the production of cDNA.

Preferably the buffer for step (a) comprises:
20-500 mM Tris pH 8.3;
10-300 mM KCl;
1-20 mM MgCl₂;
2-200 mM Tris Acetate pH 7.9;
5-500 mM Potassium Acetate;
1-10 mM Mg Acetate.

Preferably the reaction mixture for step (a) further comprises:
5-500 μg/ml Glycogen;
0.01-5% NP-40;
0.02-10 u/ml RNase Inhibitor; and
70-80 μg/ml BSA.
2-200 μM dNTPs;
0.01-100 μM oligonucleotide.

Preferably the buffer for step (b) comprises:
10-250 mM Tris pH 8.3;
5-150 mM KCl;
0.5-10 mM MgCl₂;
2-200 mM Tris Acetate pH 7.9;
5-500 mM Potassium Acetate; and
1-10 mM Mg Acetate.

Preferably the reaction mixture of step (b) further comprises:
2.5-250 μg/ml Glycogen
0.005-2.5% NP-40;
0.1-10 mM COCl₂;
1-100 μM dNTPs;
0.005-2500 μM dT24;
0.01-5 u/ml RNase Inhibitor;
35-40 μg/ml BSA;
0.05-5 mM additional dATP; and
1-500 u/ml TdT enzyme.

Preferably the buffer for step (c) comprises:
20-500 mM Tris pH 8.3;
10-300 mM KCl;
1-20 mM MgCl₂;
2-200 mM Tris Acetate pH 7.9;
5-500 mM Potassium Acetate; and
1-10 mM Mg Acetate.

Preferably the reaction mixture of step (c) further comprises:
6-7 μM Oligonucleotide;
0.1-10 mM dNTPs;
2.5-250 μg/ml Glycogen;
0.03-3.3 mM COCl₂;
0.02-1% Triton X-100;
0.005-2.5% NP-40;
35-40 μg/ml BSA;
0.05-5 mM additional dATP;
0.005 2500 μM dT24;
0.01-5 u/μl DNA Polymerase;
0.01-5 u/ml RNase Inhibitor; and
1-500 u/ml TdT enzyme.

Exonuclease digestion to produce collections of target DNA according to the invention may be performed using a suitable 3′ or 5′ exonuclease to effect degradation of the double-stranded DNA from either the 3′ or 5′ end.

Digestion with double-stranded DNA (dsDNA) exonucleases will initiate digestion at each end of a double-stranded DNA molecule such that regions of each strand that are complementary to one another are removed by digestion. Since the dsDNA exonuclease preferentially removes one strand of the double-stranded molecule digestion tends to be “self-limiting”, and will decrease when there are no remaining regions of double-stranded DNA. Thus the exonuclease treatment can effectively convert each starting double-stranded DNA molecule into two non-complementary single-stranded DNA molecules corresponding to the 3′ or 5′ “halves” of the original molecule.

With knowledge of the average size of molecules within the double-stranded DNA population (determined, for example, by gel electrophoresis) and the rate of digestion by the chosen exonuclease it is possible to chose an incubation period for digestion such that the digestion removes a chosen length of the DNA molecules. This chosen length may, for example, be approximately half the average DNA molecule length present in the starting double-stranded DNA population. Such a digest will, as with the technique described above, produce two single-stranded DNA molecules corresponding to the (3′ or 5′) “halves” of the lengths of the two starting strands of the original double-stranded DNA.

Alternatively a single strand of the double-stranded DNA may be preferentially degraded, thereby producing a single-stranded digest product. Digestion of a single strand of double-stranded DNA may be effected by incorporation of a restriction site in the double-stranded DNA to be digested. Treatment of the double-stranded molecules with a suitable restriction enzyme will yield double-stranded molecules which have a sticky end. These sticky ended double-stranded molecules may subsequently be digested, starting from either the blunt or sticky end, using an exonuclease having suitable specificity. The restriction site incorporated in the double-stranded DNA may be a naturally occurring site, or may preferably be one that has been introduced into the double-stranded molecules.

An example of an exonuclease that may be preferred for use according to the invention is exonuclease III, which is a 3′ to 5′ exodeoxyribonuclease that digests duplex DNA from a blunt end, 5′ overhang or nick.

It will be appreciated that as a result of the digestion the two remaining molecules are not complementary to one another. This therefore prevents the strands of target DNA re-hybridising to their complementary sequences found within the original double-stranded DNA population. Thus the collection of target DNA molecules are maintained in single stranded form and are therefore free to hybridise to complementary single-stranded DNA sequences in a reference sample to which they are exposed (should such sequences be present). This improves the sensitivity of the collection of target DNA molecules when used in hybridisation-based assays.

When a collection of labelled target DNA molecules according to the invention is to be used in a hybridisation-based assay it may be preferred that the arrayed DNA molecules are also treated with exonuclease in order that they too may remain single-stranded. In this case it is important to note that the target DNA and reference DNA should be treated with exonucleases having complementary specificities. For example, in one embodiment the collection of labelled target DNA molecules may be produced using a 3′-5′ exonuclease, and the reference samples treated with a 5′-3′ exonuclease. In an alternative embodiment the collection of labelled target DNA molecules may be produced using a 5′-3′ exonuclease, and the arrayed samples treated with a 3′-5′ exonuclease.

In addition to preparing total cDNA representing all expressed genes it may also be desirable to amplify and/or label a subset of the expressed genes thereby reducing the overall complexity of labelled material. The rationale behind such complexity reduction techniques is that many of the mRNAs present in a biological sample, represent transcription of so-called “house keeping” geneswhich encode products associated with the up-keep of the cell and are generally likely to be common to almostall samples. As such they represent components of gene expression patterns that may be found in both test and reference samples, but which are unlikely to be important in the development or maintenance of a biological condition or state of interest. It has been estimated that up to 65% of mRNA mass within cells may be composed of transcripts representing “house-keeping” genes. Complexity reduction techniques improve sensitivity either by simply reducing the number of individual genes represented or by specifically removing subsets of genes such as “house keeping” genes. Thus the relative abundance of those molecules representative of gene expression that remain after application of a complexity reduction technique is increased, thereby increasing the “signal to noise” ratio where the signal is produced by a single specific sequence and noise is a product all other sequences present.

A number of complexity reduction techniques may be used in effecting the method of the invention. These techniques may be used in isolation or in combination. Preferably the same complexity reduction technique, or combination of complexity reduction techniques, are used to treat the cDNA, or its derivatives, to produce both the probe library and the reference samples, although it is possible to apply complexity reduction techniques to only one of the DNA populations.

Suitable examples of complexity reduction techniques include:

Restriction Enzyme Based.

In this complexity reduction technique site specific endonucleases are used to digest the cDNA or its derivatives. Since the frequency of cleavage sites for any specific endonuclease will depend on the size and base composition of the cleavage site endonucleases can be chosen that will cut a sub-set of all DNA molecules present. For example, a restriction endonuclease recognising a six base site will, on average, cleave every 4,096 base pairs. Thus in a DNA population in which the average polynucleotide size is 2,000 bases such an endonuclease will cleave approximately half of all polynucleotides present. Following restriction digestion either the cleaved products or the uncleaved products can be selectively enriched. By choosing the appropriate restriction enzymes distinct subsets of cDNA sequences can be either eliminated or enriched. By applying this type of strategy the initial total cDNA sample can divided into subsets of genes whereby each sequence is effectively enriched making it more likely that changes in each individual gene will be detected during array hybridisation.

Thus for individual gene sequence present after applying complexity reduction there will be an increase in specific activity for each gene and an increase in the “signal to noise”.

Display Products.

Another means of selecting a subset of sequences present in the starting cDNA/mRNA population, and thereby increasing the relative abundance of each selected sequence after complexity reduction, is the use of approaches for differential cDNA display (Liang and Pardee, 1992). cDNA display selectively amplifies only those cDNA populations which contain effective priming sites for display primer(s) used. Display primers can be used to prepare distinct subsets of cDNAs directly from starting RNA (Liang and Pardee, 1992) or alternatively display amplification may be applied to amplified total cDNA populations (Candeliere et al., 1999). In essence display techniques reduce complexity by selectively enriching a subset of the sequences present in the original mRNA or cDNA population, thereby increasing the relative abundance of the selected sequences within the resultant population.

Hybridisation Depletion and Enrichment.

A variety of DNA/RNA subtraction techniques have been developed to deplete DNA/RNA sequences common to two or more pools of DNA/RNA molecules. DNA/RNA subtraction applied to DNA or RNA copies (either direct copies or amplified products) of the original extracted RNA can be used to reduce complexity by removing sequences.

Suitable DNA/RNA subtraction techniques for use according to the invention are well known. One such method involves the production of a single-stranded cDNA library (the “tracer”), such as the cDNA from which the probe library or reference samples are to be generated, from which it is desired to remove certain sequences. A collection of amplified cDNAs representing the sequences that one wishes to eliminate (the “driver”), such as housekeeping genes, is then allowed to hybridise with the tracer. Double stranded DNA molecules, representing hybrids of the tracer and the driver, may then be removed from the total population of DNA based upon their adhesion to hydroxyapatite. The remaining DNA population comprises single stranded DNA molecules representing the tracer population minus the driver population. This subtracted DNA population may then be further amplified as required.

In further refinements of this method “driver” nucleic acids may be covalently linked to compounds which facilitate the physical separation of “driver” nucleic acids (plus any annealed “tracer”) from unhybridised “tracer”. The separated populations (i.e. those sequences present only in the “tracer”, or those sequences shared by both “tracer” and “driver”) may then be enriched or depleted relative to one another. For example, driver nucleic acids may be linked to biotin, such that following hybridization all biotinylated hybrids can by segregated by interaction with immobilised avidin, allowing either subtractive enrichment or positive selection. Suitable protocols are described in Welcher et al., 1986; and Weaver et al., 1999. In alternative, but similar, approaches “driver” nucleic acids may be bound to latex beads (as described in Kuribayashi-Ohta et al., 1993, or magnetic particles (as described in Lopez-Fernandez and del Mazo, 1993; and Schraml et al. 1993.

In one embodiment hybridisation depletion/enrichment protocols can be used to remove “unwanted sequences” present in samples from which the probe library and/or reference samples are derived. The nature of the “unwanted sequences” will depend on the biological samples in question. However, as a general rule, sequences which are expressed at similar levels in diverse samples are, by their very nature, uninformative and tend simply to add to the “background” produced during hybridisation.

It is likely that genes expressed at a similar level in biologically divergent tissues will not be characteristic of a particular tissue, and will instead represent house-keeping genes. By way of example, it is unlikely that genes expressed at a similar level in tissues as biologically different as heart, lung, spleen and testes will be characteristic of any one of these tissues. Sequential hybridisation enrichment can be used to obtain a “pool” of sequences common to different tissues. The resultant pool will represent genes that contribute to the “background noise” associated with hybridisation. This pool can then be expanded and used to reduce the level of background hybridisation. For example, it is possible to subtract these common sequences from both the probe library and reference samples, thereby reducing the level of total hybridisation. Alternatively it is possible to use the pool of common genes to produce unlabelled competitor DNA and thereby reduce the level of detectable hybridisation.

Using probe libraries and reference samples produced in accordance with the techniques described above the method of the invention may be effected by reference samples and probe library under hybridising conditions. The conditions under which nucleic acids will hybridise to one another are well known to those skilled in the art. Specific conditions are described in greater detail in the accompanying Example. Further examples of conditions suitable for nucleic acid hybridisation can be found in reference works such as “Molecular Cloning: A Laboratory Manual” edited by Maniatis et al. Other suitable conditions are described in Chee et al. 1996, Iyer et al. 1999, Lipshutz et al. 1995, Lockhart et al. 1995, Schena 1996, Schena et al. 1995, Soares et al. 1997 and Southern 1996.

Similarly, methods for determining the relative degree of hybridisation between populations of nucleic acids are also well known. Methods suitable for effecting the invention include labelling of the probe iibrary with reporters such as fluorescent labels, radioactive labels or chromogenic enzymes. If the reference sample libraries are unlabelled then detection of the chosen label (after removing unbound probe) will confirm the presence of hybridisation between the sample of interest and the reference sample. Suitable techniques for labelling of the molecules comprising the probe library, for detection of hybridised probe and reference DNA molecules and for interpretation of hybridisation data are well known to those skilled in the art. These techniques include those described in Maniatis et al. 1982, Chee et al. 1996, Iyer et al. 1999, Lipshutz et al. 1995, Lockhart et al. 1995, Schena 1996, Schena et al. 1995, Soares et al. 1997 and Southern 1996.

Use of Unlabelled Competitor DNA.

When the probe library DNA is labelled and the reference sample DNA is unlabelled the sensitivity of the method of the invention may be improved by the use of unlabelled “competitor” DNA which can compete with the DNA of the probe library for hybridisation with the reference samples. The competitor DNA may be DNA representing common housekeeping genes, or it may be selected DNA representing genes common to the biological sample of interest and/or the reference samples. Since the competitor DNA is unlabelled, hybrids of competitor and reference DNA will not be detected in assessing total hybridisation.

The competitor DNA may be exposed to the reference sample DNA before the addition of the probe library DNA or at the same time as the addition of the probe library DNA. Molecules of the competitor DNA that represent genes expressed by the reference samples will then hybridise to the corresponding DNA of the reference samples. Reference sample molecules that undergo hybridisation with molecules of the competitor DNA will therefore be unable to hybridise with further molecules from the probe library. Thus by incubating the DNA of the reference samples with, for example, unlabelled competitor DNA representative of housekeeping genes it is possible to reduce the level of binding by labelled probe DNA representing the same genes. This therefore improves the sensitivity of the method of the invention since it increases the likelihood that detected hybridisation is representative of genes of interest within the samples.

Unlabelled competitor DNA representative of genes having a high frequency of expression within the biological sample of interest and/or reference samples may be generated by reverse subtraction of the DNA populations derived from the two samples.

Protocols.

The following Protocols are described with reference to Table 1. Table 1 provides details of both previously known buffers and reaction mixtures (shown in the left hand column entitled “Published”) and also novel improved buffers and reaction mixtures (shown in the right hand column entitled “Improved”).

The following protocols describe suitable methods by which labelled target DNA molecules according to the invention may be produced and used.
(a) Preparation of global amplified cDNA
   (i) Reverse transcription—Preparation of cDNA
   (ii) Terminal transferase—“Tailing”
   (iii) Global cDNA amplification
(b) Labelling of amplified DNA
   (i) Terminal Transferase labelling
   (ii) PCR labelling
(c) Complexity reduction.
   (i) Display Based
   (ii) Hybridisation depletion and enrichment
(d) Exonuclease treatment of double-stranded DNA
(e) Hybridisation of labelled DNAs.
(f) Detection of hybridisation

(g) Quantitative PCR

TABLE 1

| Published | Improved |
|---|---|
| 2X D/L | 2X D/L |
| 97.725 mM Tris HCl pH 8.3 | 97.725 mM Tris HCl pH 8.3 |
| 146.587 mM KCl | 146.587 mM KCl |
| 5.863 mM MgCl₂ | 5.863 mM MgCl₂ |
| 195 μg/ml Glycogen | 195 μg/ml Glycogen |
| 1.95% NP40 | 1.95% NP40 |
| RNAse Inhib+ | RNAse Inhib+ |
| 2.5 mM dNTPs | 1.25 mM dNTPs |
| 50 μM dT | 25 μM dT |
| 5 U/ml SUPERase- Ambion | 5 U/ml SUPERase- Ambion |
| 7.6 U/ml Prime RNase -Eppendorf | 7.5 U/ml Prime RNase - |
| | Eppendorf |
| | RT Buffer 2 + BSA |
| | 20 mM Tris Acetate pH 7.9 |
| | 50 mM K Acetate |
| | 9 mM Mg Acetate |
| | 313 μg/ml BSA |
| RT Buffer 2 | RT Buffer 2 no BSA |
| 50 mM Tris HCl pH 8.3 | 20 mM Tris Acetate pH 7.9 |
| 75 mM KCl | 50 mM K Acetate |
| 3 mM MgCl₂ | 9 mM Mg Acetate |
| 0.1 mg/ml Glycogen (Roche) | |
| 1% NP-40 (Roche) | |
| | TdT Buffer + DTT ½ CoCl₂ |
| | 20 mM Tris Acetate pH7.9 |
| | 50 mM K Ac |
| | 1 mM CoCl₂ |
| | 0.5 mM dATP |
| | 0.2 mM DTT |
| | TdT Buffer + DTT 1X CoCl₂ |
| | 20 mM Tris Acetate pH7.9 |
| | 50 mM K Ac |
| | 2 mM CoCl₂ |
| | 0.5 mM dATP |
| | 0.2 mM DTT |
| | TdT Buffer + DTT 2X CoCl₂ |
| | 20 mM Tris Acetate pH7.9 |
| | 50 mM K Ac |
| | 4 mM CoCl₂ |
| | 0.5 mM dATP |
| | 0.2 mM DTT |
| TdT Buffer | TdT Buffer no DTT |
| 200 mM potassium cacodylate pH 7.2 | 20 mM Tris Acetate pH7.9 |
| 4 mM CoCl₂ | 50 mM K Ac |
| 0.4 mM DTT | 2 mM CoCl₂ |
| 1 mM dATP | 0.5 mM dATP |

(a) Preparation of Global Amplified cDNA.

The protocol described below is based on protocols described in Brady et al. (1990) and Brady, G., and Iscove, N. N. (1993).

Suitable starting materials include total RNAs, which may be prepared from biological tissues of interest (using commercially available kits such as those manufactured by Clontech), or mRNA present in biological cells (“direct amplification”).

(i) Reverse Transcription—Preparation of cDNA.

cDNA may be prepared from the mRNA from the biological tissues according to the following protocol:
1. RNAs are adjusted to 100 microgram/ml in 10 mM Tris pH 7.5, 1 mM EDTA
2. 3 μl of each RNA is added to 3 μl “2×D/L” (Table 1).
3. Samples are heated to 65° C. for 1 minute allowed to cool to 22° C. for 3 minutes then placed on wet ice
4. After 1 to 10 minutes on ice 3 μl of “RT Buffer 2” (Table 1) containing either a combination of 85 u MMLV RTase plus 1 u AMV RTase reverse transcriptase or 1.66 units “Sensiscript™” (Qiagen).
5. Samples are Incubated 15 minutes at 37° C., heat inactivated at 65° C. for 10 minutes then cooled to 4° C.
   (ii) Terminal Transferase—‘Tailing’
1. 5 μl of each sample is mixed with 5 μl of “TdT Buffer” (Table 1) containing 2.3 units terminal transferase (Amersham Pharmacia Biotech).
2. Samples are then incubated 15 minutes at 37° C., 65° C. 10 minutes and cooled to 4° C.
   (iii) Global cDNA Amplification.

Global cDNA prepared from biological tissues according to the preceding protocols may be amplified according to the following protocol:

1. 8 μl of the tailed cDNA prepared as described above may be combined with 8 μl of:

| | |
|---|---|
| 121.4 mM | KCl |
| 8.5 mM | MgCl₂ |
| 24.25 mM | Tris-HCl pH 8.3 |
| 48 μg/ml | Glycogen (Roche) |
| 2.4% | Triton X-100 |
| 2.3 mM | dNTPs |
| 9.6 μM | Oligo Not1dT (sequence |
| | CATCTCGAGCGGCCGCTTTTTTTTTTTTTTTTTTTTTTTT) |
| 0.16 u/μl | Taq Polymerase |

2. Samples are then placed into a PCR machine and subjected to:
25 cycles
1 minute 94° C.
2 minute 42° C.
6 minute 72° C.
   followed by an additional 25 cycles:
1 minute 94° C.
1 minute 42° C.
2 minute 72° C.
3. Following completion of PCR samples are purified using the Millipore 96 well purification system (Millipore MANU 03050) following instructions provided by the manufacturer.
   (b) Labelling of Amplified DNA

The following provides suitable protocols for labelling of probe library cDNA for use according to the method of the invention. The following protocols describes the labelling of two different cDNA populations (which may be prepared using the protocols described above) with two different fluorescent markers (Cy3 and Cy5).

(i) Terminal Transferase labelling.

1. Approximately 50 ng of globally amplified cDNA of a first probe library may be added to a 20 μl reaction containing:

| | |
|---|---|
| 100 nM | FluoroLink ™ Cy3-dUTP (Amersham Pharmacia Biotech) |
| 100 mM | potassium cacodylate pH 7.2 |
| 2 mM | CoCl₂ |
| 0.2 mM | DTT |
| total 5 units | Terminal Transferase (Amersham Pharmacia Biotech) |

2. Approximately 50 ng of globally amplified cDNA of a second probe library may be added to a 20 μl reaction containing:

| | |
|---|---|
| 100 nM | FluoroLink ™ Cy5-dUTP (Amersham Pharmacia Biotech) |
| 100 mM | potassium cacodylate pH 7.2 |
| 2 mM | CoCl₂ |
| 0.2 mM | DTT |
| total 5 units | Terminal Transferase |

3. Following incubation for 1 hour at 37° C. both samples may be ethanol precipitated by the addition of:

| | |
|---|---|
| 10 μl | 7.5 M Ammonium Acetate |
| 0.5 μl | 15 mg/ml Glyco Blue (Ambion) |
| 75 μl | ethanol |

Samples may then be held on wet ice for 15 minutes, centrifuged at 4° C. at 14,000 rpm for 20 minutes and the pellets washed twice with 70% ethanol, allowed to dry 10 minutes at room temperature then resuspended in 5 μl 10 mM Hepes pH 7.5, 1 mM EDTA.

(ii) PCR Labelling.

Further rounds of PCR amplification can be used to incorporate fluorescent markers directly or indirectly coupled to nucleotides present in the PCR reaction. An example of such an approach is given below.

1. Approximately 0.5 ng of globally amplified cDNA of a first probe library may be added to a 20-100 μl reaction containing:

| | |
|---|---|
| 100 nM | FluoroLink ™ Cy3-dUTP |
| | (Amersham Pharmacia Biotech) |
| 100 nM | dNTPs |
| 1 μM | Oligo Not1dT (sequence |
| | CATCTCGAGCGGCCGCTTTTTTTTTTTTTTTTTTTTTTTT) |
| 16 mM | (NH₄)₂SO₄ |
| 67 mM | Tris-HCl (pH 8.8 at 25° C.) |
| 0.01% | Tween-20 |
| 0.16 u/μl | Taq Polymerase |

2. Approximately 0.5 ng of globally amplified cDNA of a second probe library may be added to a 20-100 μl reaction containing:

| | |
|---|---|
| 100 nM | FluoroLink ™ Cy5-dUTP |
| | (Amersham Pharmacia Biotech) |
| 100 nM | dNTPs |
| 1 μM | Oligo Not1dT (sequence |
| | CATCTCGAGCGGCCGCTTTTTTTTTTTTTTTTTTTTTTTT) |
| 16 mM | (NH₄)₂SO₄ |
| 67 mM | Tris-HCl (pH 8.8 at 25° C.) |
| 1.5 mM | MgCl₂ |
| 0.01% | Tween-20 |
| 0.16 u/μl | Taq Polymerase |

3. Both samples are then placed into a PCR machine and subjected to:

25 cycles

| | |
|---|---|
| 30 seconds | 94° C. |
| 1 minute | 42° C. |
| 2 minutes | 72° C. |

4. Following completion of the PCR step both samples may be stored at −20° C. for further processing or ethanol precipitated by the addition of:

| | |
|---|---|
| 0.5 original sample volume | 7.5 M Ammonium Acetate |
| 0.025 original sample volume | 15 mg/ml Glyco Blue (Ambion) |
| 3.5 original sample volumes | ethanol |

Samples may then be held on wet ice for 15 minutes, centrifuged at 4° C. at 14,000 rpm for 20 minutes and the pellets washed twice with 70% ethanol, allowed to dry 10 minutes at room temperature then resuspended in 5 μl 10 mM Hepes pH 7.5, 1 mM EDTA.

(c) Complexity Reduction.

Double stranded DNA, such as cDNA, suitable for use according to the invention may advantageously be subjected to complexity reduction techniques, in order to exclude DNA not thought to be of interest, before exonuclease digestion.

There are many possible complexity reduction techniques that are suitable for use with the method of the invention.

(i) Display Based

The following protocol is suitable for effecting a “display products” complexity reduction technique according to the method of the invention. The protocol provides for the preparation of two different amplified cDNA populations from the same cDNA population (“total cDNA”).

Selected subsets of cDNA within a global amplified total cDNA population may be further amplified based on protocols described in:

Candeliere, G. A., Rao, Y., Floh, A., Sandier, S. D., and Aubin, J. E. (1999). cDNA fingerprinting of osteoprogenitor cells to isolate differentiation stage-specific genes. Nucleic Acids Research 27, 1079-83.

A suitable protocol is as follows:
1. Purified globally amplified total cDNA prepared as described above may be diluted 100 fold in 2 mM Tris pH 7.5, 0.2 mM EDTA.

2. Two separate subsets of cDNAs may then be selectively amplified from the total cDNA by separately adding 10 μl of total cDNA to 10 μl of PCR mixture A and 10 μl of total cDNA to 10 μl of PCR mixture B. and subjecting both to:

2 cycles as follows:

| | | |
|---|---|---|
| 94° C. | 1 | minutes; |
| 35° C. | 3 | minutes; |
| 72° C. | 3 | minutes; |

followed by 30 cycles as follows:

| | | |
|---|---|---|
| 94° C. | 30 | seconds; |
| 50° C. | 30 | seconds; |
| 72° C. | 1 | minute; and |

1 cycle as follows:

| | | |
|---|---|---|
| 72° C. | 5 | minutes. |

PCR mixture A

| | |
|---|---|
| 25 μM | Display Oligo A - |
| | CAGCCAGTCTTGAGGCAACACC |
| 0.5 μM | dNTPs (Sigma) |
| 32 μM | (NH₄)₂SO₄ |
| 134 μM | Tris-HCl (pH 8.8 at 25° C.) |
| 0.01% | Tween-20 |
| 3 μM | MgCl₂ |
| 25 u/ml | Taq Polymerase |

PCR mixture B

| | |
|---|---|
| 25 μM | Display Oligo B - CCAGCAAGAGCACAAGAGGAAGAG |
| 0.5 mM | dNTPs (Sigma) |
| 32 mM | (NH₄)₂SO₄ |
| 134 mM | Tris-HCl (pH 8.8 at 25° C.) |
| 0.01% | Tween-20 |
| 3 mM | MgCl₂ |
| 25 u/ml | Taq Polymerase |

Following PCR all samples may be purified using GFX purification columns (Amersham Pharmacia) following the manufacturer's instructions.

(ii) Hybridisation Depletion and Enrichment

The term driver refers to the cDNA used to deplete and/or enrich in the tracer cDNA population. The resultant depleted or enriched sequences will be derived from the tracer cDNA population. In the following examples all driver cDNAs are prepared in PCR reactions containing dUTP (not dTTP) to allow removal of residual driver cDNAs using the dUTP specific UNG nuclease.

Based on Methods Described in:

Analysis of gene-expression in a complex differentiation hierarchy by global amplification of cdna from single cells. Brady, G, Billia F, Knox J, Hoang T, Kirsch I R, Voura E B, Hawley R G, Cumming R, Buchwald M, Siminovitch K, Miyamoto N, Boehmelt G, and Iscove N N: Current Biology 1995, 5: 909-922.

Foot, H C C, Brady G, and Franklin F C H. (1996). Subtractive Hybridisation. In Plant Molecular Biology Laboratory Manual, M. Clark, ed. (London: Springer Verlag).

Weaver, D L, Núñez C, Brunet C, Bostock V, and Brady G. (1999). Single-cell RT-PCR cDNA subtraction. In Molecular Embryology: Methods and Protocols., P. Sharpe and I. Mason, eds. (Totowa, N.J., USA: Humana Press), pp. 601-609.

Depletion/Subtraction

1. Preparation of Tracer and Driver:

Tracer

Approximately 0.5 ng of globally amplified cDNA added to a 20-100 μl reaction containing:

| | |
|---|---|
| 250 nM | dATP, dTTP, dCTP, dGTP |
| 1 μM | Oligo Not1dT (sequence |
| | CATCTCGAGCGGCCGCTTTTTTTTTTTTTTTTTTTTTTTT) |
| 16 mM | (NH₄)₂SO₄ |
| 67 mM | Tris-HCl (pH 8.8 at 25° C.) |
| 1.5 mM | MgCl₂ |
| 0.01% | Tween-20 |
| 0.16 u/μl | Taq Polymerase |

Driver

Approximately 0.5 ng of globally amplified cDNA added to a 20-100 μl reaction containing:

| | |
|---|---|
| 250 nM | dATP, dUTP, dCTP, dGTP |
| 1 μM | Oligo Not1dT (sequence |
| | CATCTCGAGCGGCCGCTTTTTTTTTTTTTTTTTTTTTTTT) |
| 16 mM | (NH₄)₂SO₄ |
| 67 mM | Tris-HCl (pH 8.8 at 25° C.) |
| 1.5 mM | MgCl₂ |
| 0.01% | Tween-20 |
| 0.16 u/μl | Taq Polymerase |

Both tracer and driver are then placed into a PCR machine and subjected to:

25 cycles

| | |
|---|---|
| 30 seconds | 94° C. |
| 1 minute | 42° C. |
| 2 minutes | 72° C. |

Following completion of the PCR reaction both tracer and driver cDNAs are then purified using commercial purification systems such as GFX (Amersham Pharmacia).

Biotinylation of Driver.

Place 20-50 μl driver DNA (2-50 μg) in a 1.5 ml screw-cap tube. Boil for 2 minutes and place directly on ice in a small ice tray+rack.

Add 20 μl 2 mg/ml photobiotin to the DNA and mix well. With the lids left off place the tubes upright on ice 10 cm from the bulb and irradiate for a total 10 minutes. After the first 5 minutes remove the tubes from under the light source (avoid direct irradiation), flick the tube to mix and replace under the light source for the remaining 5 minutes.

Remove the sample (avoid direct irradiation) and mix in the remaining 20 μl of photobiotin and place under the light for an additional 5 minutes.

Add 1/10th volume of 1M Tris-Cl, pH 8.0 to stop the reaction.

Purify using commercial purification systems such as GFX (Amersham Pharmacia).

2. Hybridisation of Tracer Plus Driver and Tracer Enrichment:

To a 0.5 ml tube add and mix in this order:

| | |
|---|---|
| 0.5 μg | tracer DNA |
| 10 μg | biotinylated driver DNA |

adjust volume to 20 μl with water then add:

| | |
|---|---|
| 8 μl | 5xHyb GEH |
| 12 μl | 40% PEG |
| Heat sample: | 5 minutes 98° C., |
| | 5 minutes at 80° C. |
| | 7 minutes at 74° C. |
| | 60 minutes at 68° C. |
| | then hold at 68° C. while seperating |
| | biotinylated molecules |

Remove biotinylated molecules using avidin bound to a solid support. In practise this can be carried out using commercial products as ditrected by the manufacturer such as Streptavidin Magnasphere™ Paramagnetic particles (SA-PMPs) provided by Promega.

Following removal of biotinylated molecules the remaining tracer can be subjected to further rounds of subtraction by addition of fresh biotinylated driver DNA and repeating the process described above. Typically three sequential rounds of subtraction are used but additional rounds may be added if required.

The final depleted product is then amplified using PCR conditions described for the original tracer amplification.

5xHyb GEH

| | |
|---|---|
| 90 mM | EPPS pH 8.5 |
| 10 mM | EDTA pH 8.0 |
| 0.5% | Triton X-100 |
| 3.75 M | NaCl |

Negative Subtraction or Attraction
1. Preparation of Tracer and Driver:
Tracer

Approximately 0.5 ng of globally amplified cDNA added to a 20-100 μl reaction containing:

| | |
|---|---|
| 250 nM | dATP, dTTP, dCTP, dGTP |
| 1 μM | Oligo Not1dT (sequence |
| | CATCTCGAGCGGCCGCTTTTTTTTTTTTTTTTTTTTTTTT) |
| 16 mM | (NH₄)₂SO₄ |
| 67 mM | Tris-HCl (pH 8.8 at 25° C.) |
| 1.5 mM | MgCl₂ |
| 0.01% | Tween-20 |
| 0.16 u/μl | Taq Polymerase |

Driver

Approximately 0.5 ng of globally amplified cDNA added to a 20-100 μl reaction containing:

| | |
|---|---|
| 250 nM | dATP, dUTP, dCTP, dGTP |
| 1 μM | Oligo Not1dT (sequence |
| | CATCTCGAGCGGCCGCTTTTTTTTTTTTTTTTTTTTTTTT) |
| 16 mM | (NH₄)₂SO₄ |
| 67 mM | Tris-HCl (pH 8.8 at 25° C.) |
| 1.5 mM | MgCl₂ |
| 0.01% | Tween-20 |
| 0.16 u/μl | Taq Polymerase |

Both tracer and driver are then placed into a PCR machine and subjected to:

| 25 cycles | |
|---|---|
| 30 seconds | 94° C. |
| 1 minute | 42° C. |
| 2 minutes | 72° C. |

Following completion of the PCR reaction both tracer and driver cDNAs are then purified using commercial purification systems such as GFX (Amersham Pharmacia).

Biotinylation of Driver

Place 20-50 μl driver DNA (2-50 μg) in a 1.5 ml screw-cap tube. Boil for 2 minutes and place directly on ice in a small ice tray+rack.

Add 20 μl 2 mg/ml photobiotin to the DNA and mix well. With the lids left off place the tubes upright on ice 10 cm from the bulb and irradiate for a total 10 minutes. After the first 5 minutes remove the tubes from under the light source (avoid direct irradiation), flick the tube to mix and replace under the light source for the remaining 5 minutes.

Remove the sample (avoid direct irradiation) and mix in the remaining 20 μl of photobiotin and place under the light for an additional 5 minutes.

Add 1/10th volume of 1M Tris-Cl, pH 8.0 to stop the reaction.

Purify using commercial purification systems such as GFX (Amersham Pharmacia).

2. Hybridisation of Tracer Plus Driver and Tracer Enrichment:

To a 0.5 ml tube add and mix in this order:

| | | |
|---|---|---|
| 0.5-10 | μg | tracer DNA |
| 10 | μg | biotinylated driver DNA 1 |

adjust volume to 20 μl with water then add:

| | | |
|---|---|---|
| 8 | μl | 5xHyb GEH |
| 12 | μl | 40% PEG |

```
Heat sample:
  5 minutes 98° C.,
  5 minutes at 80° C.
  7 minutes at 74° C.
60 minutes at 68° C.
then hold at 68° C. while seperating biotinylated molecules
```

Remove biotinylated molecules using avidin bound to a solid support. In practise this can be carried out using commercial products as ditrected by the manufacturer such as Streptavidin Magnasphere™ Paramagnetic particles (SA-PMPs) provided by Promega.

Release tracer DNA bound to driver DNA 1 by denaturing the driver DNA 1/tracer DNA hybrids. For examples using SA-PMPs the washed SA-PMPs and their attendant driver DNA1/tracer DNA hybrids can be heated to 96° C. to release tracer DNA and bound driver DNA 1 removed by magnetic attraction of the SA-PMPs.

Released tracer DNA can then be subjected to further rounds of attraction by repeating the process with separate drivers (driver DNAs 2, 3, 4 etc).

The final “attracted” product will be enriched for sequences common to all driver DNAs used and can be amplified using PCR conditions described for the original tracer amplification.

5xHyb GEH

| | |
|---|---|
| 90 mM | EPPS pH 8.5 |
| 10 mM | EDTA PH 8.0 |
| 0.5% | Triton X-100 |
| 3.75 M | NaCl |

(d) Exonuclease Treatment of Double-Stranded DNA.

Note exonuclease treatment can be applied to freshly amplified cDNA, labelled cDNA or cDNA that has been subjected to a complexity reduction technique.

(i) 3′-5′-Exonuclease—Exonuclease III

1. Add 100 μl Freshly Amplified cDNA (Section bii Step 3) to 5 μl of a Exonuclease III Buffer Consisting of:

| | |
|---|---|
| 660 mM | Tris pH 8.0 |
| 66 mM | MgCl₂ |
| 50 mM | DTT |
| 500 μg/ml | BSA |

For digestion add 10 units Exonuclease III (Amersham Pharmacia) for negative controls omit enzyme.

2. Incubate 15 Minutes at 37° C.

3. Following Heat Inactivation at 75° C. for 10 Minutes Ethanol Purify Using SigmaSpin Columns (Sigma) and Dried Down (Lyophilised).

(ii) 5′-3′-Exonuclease—T7 Gene 6 Exonuclease

1. Add Freshly 0.5-5 μg cDNA to a 50 μl Reaction Consisting of:

| | |
|---|---|
| 40 mM | Tris pH7.5 |
| 20 mM | MgCl₂ |
| 50 mM | NaCl |
| 10 units | T7 Gene 6 Exonuclease (Amersham Pharmacia) |

2. Incubate 30 Minutes at 37° C.

3. Following Heat Inactivation at 75° C. for 30 Minutes Ethanol Precipitate by the Addition of:

| | |
|---|---|
| 0.5 original sample volume | 7.5 M Ammonium Acetate |
| 0.025 original sample volume | 15 mg/ml Glyco Blue (Ambion) |
| 3.5 original sample volumes | ethanol |

Samples may then be held on wet ice for 15 minutes, centrifuged at 4° C. at 14,000 rpm for 20 minutes and the pellets washed twice with 70% ethanol, allowed to dry 10 minutes at room temperature then resuspended in 5 μl 10 mM Hepes pH 7.5, 1 mM EDTA.

(e) Hybridisation of Labelled DNAs.

Hybridisation of probe library and reference samples according to the method of the invention may be effected as follows, using an array and probe libraries prepared as described above.
1. An array slide may be prehybridised at 42° C. for 1 hour in the following buffer:
50% Formamide
5×SSC
0.1% SDS
10 mg/ml BSA
2. The array slide may then be washed four times with H₂O and once in Isopropanol and dried 5 minutes at room temperature.

3. The following mixture may then be prepared:

| | |
|---|---|
| 50% v/v | Formamide |
| 5X | SSC |
| 0.1% | SDS |
| 0.5 mg/ml | Poly A RNA |
| 0.5 mg/ml | Yeast tRNA |
| 0.5 mg/ml | Salmon Sperm DNA (10-30 ug) |
| 50 ug/ml | Cot1 DNA |

combined Cy3 and Cy 5 probes
(Total volume 45 μl)

4. The mixture may then be heated at 95° C. for 5 minutes and chilled on wet ice 3 minutes.
5. The mixture may be applied to a cover slip and the pre-warmed (42° C.) array slide (arrayed material facing downwards) lowered onto cover slip to the point when it is just possible to lift the cover slip up with surface tension.
6. The slide may be placed into a moisturised slide hybridisation chamber and incubated 42° C. o/n. (<16 hr).
7. Following hybridisation the entire slide may be immersed in 2×SSC and the cover slip removed.
8. The exposed slide may then be washed twice 2×SSC/0.1% SDS (5 minutes RT each wash) followed by 2 washes with 2×SSC (5 minutes RT each wash) and drying at room temperature.
   (f) Detection of Hybridisation.

The following protocol is suitable for detection and analysis of hybridisation in the method of the invention.
1. Scanning of the slide and quantification of red (Cy5 635 nm) and green (Cy3 532 nm) fluorescence may be carried out using a GenePix 4000b as recommended by the manufacturer.
2. Following scanning data may be analysed using commercially available software.
   (g) Quantitative PCR.

Quantitative PCR measurements were made using the “qPCR™ core kit for Sybr® Green I” (Eurogentech) as recommended except 25 μl reaction volumes were used. For amplification using the NotldT oligo a final oligo concentration of 4.75 μM was used and for gene-specific PCR a final concentration of 0.5 μM was used. Real-time measurements were made using an Applied Biosystems “ABI Prism® 7000 Sequence Detection System” following the manufacturers recomendations. PCR conditions for

Not1dT oligo PCR reactions were:
40 cycles

| | |
|---|---|
| 95° C. | 15 seconds |
| 42° C. | 30 seconds |
| 72° C. | 1 minute |

PCR conditions for gene-specific PCR reactions were:
1 cycle

| | |
|---|---|
| 50° C. | 2 minutes |

1 cycle

| | |
|---|---|
| 95° C. | 10 minutes |

40 cycles

| | |
|---|---|
| 95° C. | 15 seconds |
| 60° C. | 1 minute |

Improved Reagents Protocol for the Preparation of cDNA.

The Protocols listed above illustrate suitable methods by which global amplified cDNA may be produced for use according to the invention. However, the following provide an improved set of reagents and improved protocol that may be used in the preparation of cDNA as an alternative to the Protocol described in Section (a) above.

The following reagents and protocol increase the efficiency, accuracy and yield of cDNA produced. A kit for carrying out the method of the invention may comprise reagents/compositions as defined in A1 to A9 and E1 to E3 below.

Reagents:

A1 RT Buffer 1

97.725 mM Tris HCl pH 8.3
146.587 mM KCl
5.863 mM MgCl₂
195 μg/ml Glycogen
1.95% NP40
   A2 RNAse Inhibitors
1.25 mM dNTPs
25 μM dT
5 U/ml SUPERase-Ambion
7.5 U/ml Prime RNase-Eppendorf
   A3 RT Buffer 2
20 mM Tris Acetate pH 7.9
50 mM K Acetate
9 mM Mg Acetate
313 μg/ml BSA
   A4 TdT Buffer
20 mM Tris Acetate pH7.9
50 mMKAc
   A5 CoCl₂
2 mM CoCl₂
   A6 dATP
0.5 mM dATP
   A7 Taq Buffer
143.76 mM KCl
30 mM Tris HCl pH ˜8.5
10 mM MgCl2
56 μg/ml Glycogen
0.3% Triton
   A8 dNTPs
dNTPs 25 mM each
   A9 Oligo
297 LM NotldT
(CATCTCGAGCGGCCGCT24)
   E1 RTMMLV RTase 178 u/μl
AMV RTase 2.2 u/μl
   E2 TdT
14 u/μl terminal transferase
   E3 Taq Polymerase
5 u/μl Taq polymerase
   Protocol:

Prior to starting thaw all reagents on ice, mix well and briefly spin.

Reverse Transcriptase

1. Make up fresh 1st Strand Buffer as follows:

| | | |
|---|---|---|
| 50 μL | A1 | RT Buffer 1 |
| 1.6 μL | A2 | RNAse Inhib+ |

2. Mix 2 μl RNA sample with 2 μl 1st Strand Buffer heat 65° C. for 1 minute and cool at room temperature for 3 minutes then place on ice.

3. Make up fresh RT Solution as follows:

| | | |
|---|---|---|
| 50 μL | A3 | RT Buffer 2 |
| 1.6 μL | E1 | RT |

4. Add 2 μl RT Solution to each sample and incubate 15 minutes at 37° C., heat inactivate at 65° C. for 10 minutes then place on ice.
   Tailing

5. Prepare Tailing Mix as follows:

| | | |
|---|---|---|
| 137.25 μL | A4 | TdT Buffer |
| 12 μL | A5 | CoCl₂ |
| 0.75 μL | A6 | dATP |
| 5 μL | E2 | TdT |

6. Add 6 μL Tailing Mix to all samples, incubate 15 minutes at 37° C., heat inactivate at 65° C. for 10 minutes then place on ice.
   PCR

7. Prepare PCR Mix as follows:

| | | |
|---|---|---|
| 487.5 μL | A7 | Taq Buffer |
| 52 μL | A8 | dNTPs |
| 17.7 μL | A9 | Oligo |
| 17.7 μL | E3 | Taq Polymerase |

8. Add 24 μL PCR Mix to all samples, mix and subject to the following PCR protocol:
95° C.-30 seconds
42° C.-2 minutes
72° C.-6 minutes×6 cycles
   followed by:
95° C.-15 seconds
42° C.-30 seconds
72° C.-1 minute×34 cycles
   NOTE: Use a PCR machine fitted with a heated lid to avoid evaporation.
9. Purify using either the Millipore 96 well purification system (Millipore MANU 03050) or the GFX™ PCR DNA and Gel Band Purification Kit (Amersham).

### EXPERIMENTAL RESULTS

1) Increased Sensitivity of Exonuclease Derivative Target DNA.

The following experimental results illustrate the increased sensitivity of labelled target DNA in accordance with the invention.

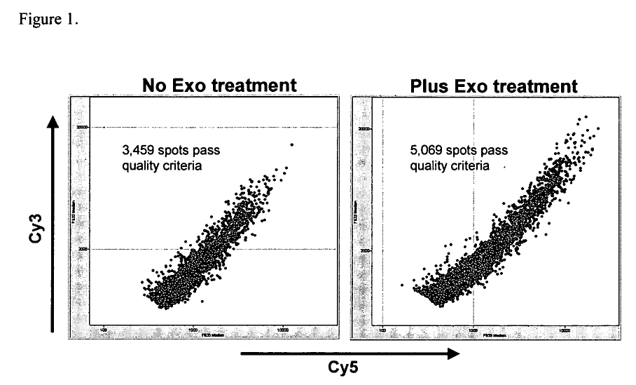

FIG. 1 illustrates that exonuclease treated target DNA has greater sensitivity than non-exonuclease treated target DNA.

FIG. 1 shows the results of hybridising a glass slide gene array of individual mouse gene sequences with fluorescently labelled global cDNAs. RNAs were prepared from colonic epithelium from a wild-type (WT) mouse and a transgenic mouse lacking a checkpoint gene (KO) using “RNA aqueous™” (Ambion). Global amplified cDNA was prepared using 10 ng of either WT or KO total RNA according to the Protocols above (Section a). PCR labelling according to the Protocols (Section bii) was then applied to produce KO global cDNA fluorescently labelled with Cy3, and WT global cDNA fluorescently labelled with Cy5.

Immediately following the labelling step equal volumes of the KO global cDNA fluorescently labelled with Cy3 and WT global cDNA fluorescently labelled with Cy5 were mixed together and combined with Exonuclease III buffer (Protocol section ci step 1). The combined sample was then divided in two equal volumes and Exonuclease III added to one half. After incubation and purification both halves (+/−Exonuclease III digestion) were used as purified probes hybridise to and analyse duplicate glass slide gene arrays of individual mouse gene sequences, as described in the Protocols above (Sections dande).

The left hand panel of the Figure illustrates the extent of hybridisation to the slide gene array of target DNA that had not been subjected to exonuclease digestion. The right hand panel illustrates the extent of hybridisation to the slide gene array of target DNA that had been subject to exonuclease digestion.

In both the left and right hand panels the extent of KO global cDNA hybridisation (detected as Cy3 fluorescence) and WT global cDNA hybridisation (detected as Cy5 fluorescence) to individual mouse DNA sequences (each represented as a single spot in the figure) is plotted on an identical logarithmic scale. It can clearly be seen that the extent of hybridisation is markedly increased following exonuclease digestion (indicated by an extended spread toward the upper right hand corner, seen in the left hand panel). Furthermore, target DNA which is an exonuclease derivative of double-stranded DNA generated 5069 detectable hybridisation events which passed the quality control criteria used by the analysis software GenePix 4.0 whereas using the same criteria only 3459 hybridisation events which passed the quality control criteria produced by its non-digested counterpart. This illustrates that the exonuclease digestion increases the sensitivity of the target DNA.

2) Improved Preparation of cDNA.

The success of the global amplification process relies on three successive enzymatic steps:
i) reverse transcription (to produce a global cDNA population);
ii) terminal transferase “tailing” (to extend the molecules of the global cDNA); and
iii) polymerase chain reaction (to amplify the tailed global cDNA).

These steps are carried out by simple addition of a buffer/enzyme mixture to the reaction mixture of the preceding step.

Maintaining effective enzymatic performance for each step is problematic when the standard reverse transcriptase, terminal transferase and Taq polymerase buffers are used because they are very different from one-another. Although the original, previously published, buffer conditions are capable of providing efficient amplification (refs) the sequential nature of the three combined reactions has meant that minor changes in the buffer set which are often inadvertently introduced in preparing new batches of apparently identical buffers, can reduce the overall efficiency of the whole amplification procedure.

This variability in the original buffers has led some to introduce additional steps or purifications to the original amplication procedure (refs). Although, these additions allow efficient amplification the use of the additional steps increases the likelihood of error and/or contamination, as well as increasing the “hands-on” time required by a user, when compared to the original simple one tube method.

We have developed a new set of buffers which overcome the variability inherent in the buffers used for the original method while maintaining the simple one-tube nature of the original method. We have changed the overall buffer base to included acetate buffers since these buffers are more representative of cellular conditions and have provided a broad buffer system for restriction enzymes (McClellan et al 1988). In addition we have also optimised the concentrations of dNTPS, CoCl₂, BSA and DTT to provide a radically new set of buffers which together provide a dramatic improvement over the previous published buffers.

i) Optimisation of dNTP and Oligonucleotide Concentration to Improve cDNA Yield and Accuracy.

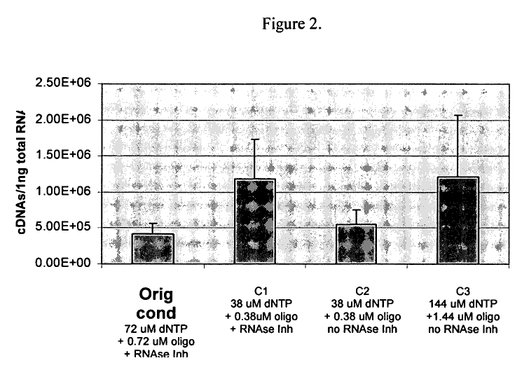

FIG. 2 illustrates that varying the concentration of dNTPs and oligonucleotides present in the reaction mixture during the reverse transcription step of global cDNA amplification significantly alters the yield of cDNA.

For all conditions tested the reverse transcriptase and terminal transferase reactions as described in the global amplification protocol (Protocols Section (a) i and ii) were applied to triplicate sets of 1 ng total mouse spleen RNA (Ambion). For each reverse transcriptase reaction 1.66 units “Sensiscrip™” (Qiagen) was used. All buffers used were as outlined in the left hand (“Published”) column of Table I with the exception of “RNAse Inhib+” which was adjusted to provide the final concentrations shown in FIG. 2.

Following completion of the terminal transferase step quantitative measurements of total cDNA yield were made by removing 0.5 μL material and applying quantitative PCR using the oligo Not1dT as described in the Protocols (Section g).

The left hand bar of FIG. 2 (marked “orig cond”) shows cDNA yield using previously published conditions (72 μM dNTP; 0.72 μM oligonucleotide; and RNase inhibitors).

The bar marked C1 shows cDNA yield using 38 μM dNTP; 0.38 μM oligonucleotide; and 0.37 u/ml RNase inhibitors. These conditions cause a near threefold improvement in cDNA yield as compared to previously published methods.

The bar marked C2 shows cDNA yield using 38 μM dNTP; 0.38 μM oligonucleotide; and no RNase inhibitors. It can be seen that cDNA yield is much reduced as compared to the conditions used in C1.

The bar marked C3 shows cDNA yield using 144 μM dNTP; 1.44 μM oligonucleotide; and no RNase inhibitors. While these conditions appear to give a high yields of cDNA the amplification plots were found to be irregular when compared to those obtained using the other conditions.

In conclusion it can be seen that performing the reverse transcription step of cDNA preparation in the presence of 38 μM dNTP; 0.38 μM oligonucleotide; and 0.37 u/ml RNase inhibitors significantly increases cDNA yield.

ii) Improved cDNA Yield Using Acetate Buffers.

For all conditions tested the reverse transcriptase and terminal transferase reactions as described in the global amplification protocol (Protocol section (a) i and ii) were applied to triplicate sets of 1 ng total mouse spleen RNA (Ambion). For each reverse transcriptase reaction 1.66 units “Sensiscript™” (Qiagen) was used.

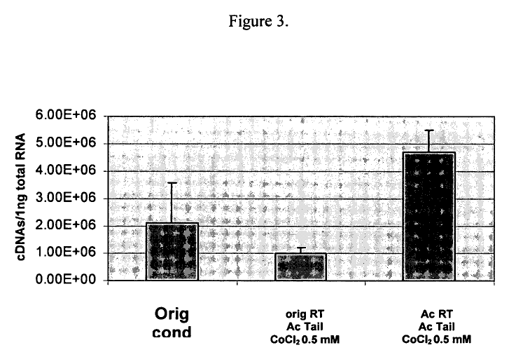

For the first column shown in FIG. 3 all buffers used were as outlined in the “Published” column of Table 1.

For the second column shown in FIG. 3 all buffers used were as described in the “Published” column of Table 1 with the exception of that the “TdT Buffer+DTT ½ CoCl₂” described in the right hand (“Improved”) column of Table I was used for the terminal transferase step.

For the third column shown in FIG. 3 all buffers used were as described in the “Published” column of Table I with the exception of that the “RT Buffer 2 no BSA” was used in the reverse transcriptase step and “TdT Buffer+DTT ½ CoCl₂” was used for the terminal transferase step (both shown in right hand column of Table 1).

Following completion of the terminal transferase step quantitative measurements of total cDNA yield were made by removing 0.5 μL material and applying quantitative PCR using the oligo NotldT as described in Protocol section (g).

The increase seen in the third column illustrates the improve cDNA yield produced when both the reverse transcription step and the homopolymer tailing step are carried out in the presence of acetate buffers. It can be seen that the use of acetate buffers during these steps increases yield significantly (two fold) as compared to previously published conditions. The reduction of cDNA yield seen in the middle column illustrates the importance of the combinatorial aspect of the sequential buffer system.

iii) Optimisation of concentration of CoCl₂, To assess the importance of CoCl₂ concentration following the switch to an acetate buffer system the reverse transcriptase and terminal transferase reactions as described in the global amplification protocol (Protocols Section (a) i and ii) were applied to triplicate sets of 1 ng total mouse spleen RNA (Ambion) using the buffers described below. For each reverse transcriptase reaction 1.66 units “Sensiscript™” (Qiagen) was used.

Following completion of the terminal transferase step quantitative measurements of total cDNA yield were made using the oligo NotldT as described in Protocols Section (g).

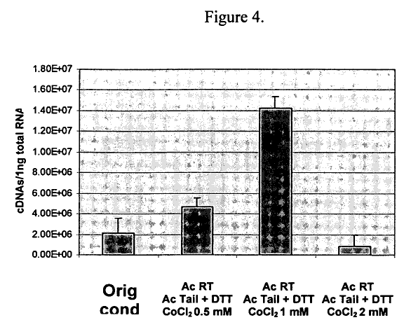

For the first column shown in FIG. 4 all buffers used were as outlined in the “Published” column of Table 1. For the columns 2-3 shown in FIG. 4 the buffers used were as outlined in the “Improved” column of Table 1. For the second column the “TdT Buffer+DTT ½ CoCl₂” was used to give a final CoCl₂ concentration of 0.5 mM, for the third column the “TdT Buffer+DTT 1×CoCl₂” was used to give a final CoCl₂ concentration of 1 mM and for the fourth column the “TdT Buffer+DTT 2×CoCl₂” was used to give a final CoCl₂ concentration of 2 mM.

As can be seen the use of 1 mM CoCl₂ provides significant benefits compared to 0.5 mM CoCl₂ and 2 mMCoCl₂.

iv) cDNA Yield is Increased when Reverse Transcription is Performed in the Presence of Bovine Serum Albumen.

To assess the importance of bovine serum albumen (BSA) following the switch to an acetate buffer system the reverse transcriptase and terminal transferase reactions as described in the global amplification protocol (Protocol section (a) i and ii) were applied to triplicate sets of 1 ng total mouse spleen RNA (Ambion) using the buffers described below. For each reverse transcriptase reaction 1.66 units “Sensiscript™” (Qiagen) was used.

Following completion of the terminal transferase step quantitative measurements of total cDNA yield were made by removing 0.5 μL material and applying quantitative PCR using the oligo NotldT as described in Protocol section (g).

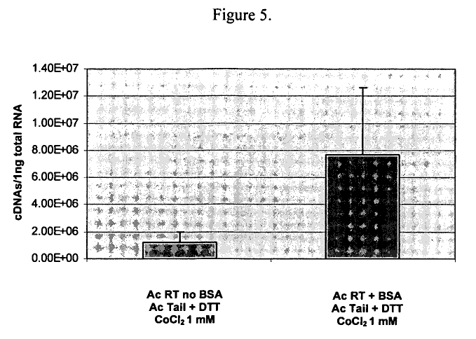

For the first column in FIG. 5 the buffers used were as outlined in the “Improved” column of Table 1 using “RT Buffer 2 no BSA” for the reverse transcriptase step and “TdT Buffer+DTT 1×CoCl₂” for the terminal transferase step.

For the second column in FIG. 5 the buffers used were as outlined in the “Improved” column of Table 1 using “RT Buffer 2+BSA” for the reverse transcriptase step and “TdT Buffer+DTT 1×CoCl₂” for the terminal transferase step.

The conditions, producing the cDNA yield illustrated by the left and right hand bars, differ only in the presence or absence of BSA during the reverse transcription reaction.

It can be seen that the presence of BSA, as illustrated in the right hand column, significantly increases cDNA yield when compared to the absence of BSA, as shown in the left hand column.

v) Absence of Dithiothreitol (DTT) During Homopolymer Tailing Step Increases cDNA Yield.

DTT is a component of previously reported reaction mixtures for use in the preparation of cDNA. To assess the importance of DTT following the switch to an acetate buffer system the reverse transcriptase and terminal transferase reactions as described in the global amplification protocol (Protocol section (a) i and ii) were applied to triplicate sets of 1 ng total mouse spleen RNA (Ambion) using the buffers described below. For each reverse transcriptase reaction 1.66 units “Sensiscript™” (Qiagen) was used.

Following completion of the terminal transferase step quantitative measurements of total cDNA yield were made by removing 0.5 μL material and applying quantitative PCR using the oligo NotldT as described in Protocol section (g).

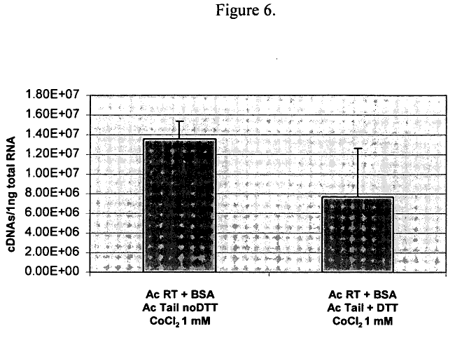

For the first column in FIG. 6 the buffers used were as outlined in the “Improved” column of Table 1 using “RT Buffer 2+BSA” for the reverse transcriptase step and “TdT Buffer no DTT” for the terminal transferase step.

For the second column in FIG. 6 the buffers used were as outlined in the “Improved” column of Table 1 using “RT Buffer 2+BSA” for the reverse transcriptase step and “TdT Buffer+DT 1×CoCl₂” for the terminal transferase step.

Since the reaction conditions differ only in the omission or inclusion of DTT the results illustrate that removal of DTT improves the level of amplification achieved.

vi) Comparison of cDNA Yield Using Previously Reported Preparation Conditions, and cDNA Yield Using Improved Preparation Conditions.

To assess the cumulative effect of the improved buffers described on cDNA yield, reverse transcriptase, terminal transferase and PCR reactions as described in the global amplification protocol (Protocol section (a) i, ii and iii) were applied to triplicate sets of 1 ng total mouse spleen RNA (Ambion) using the buffers set out below.

For each reverse transcriptase reaction 1.66 units “Sensiscript™” (Qiagen) was used. Following addition of the PCR buffer (Protocol section (a) iii), and prior to initiation of PCR cycling, 2 μL of each reaction was set aside and the remaining sample subjected to a limited PCR of 6 cycles only.

Samples of cDNA produced by each protocol used were subjected to quantitative PCR using the oligonucleotide NotldT as described in Protocol section (g). Quantitative PCR was applied to samples taken prior to and after the 6 cycles PCR.

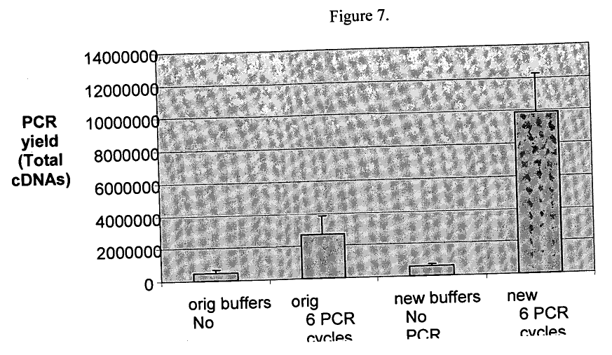

For all samples labelled “orig buffers” in FIG. 7 the buffers used were as outlined in the “Published” column of Table 1.

For all samples labelled “new buffers” in FIG. 7 the buffers used were outlined in the “Improved” column of Table 1, using the specific reverse transcriptase step and terminal transferase buffers “RT Buffer 2+BSA” and “TdT buffer no DTT”.

Samples labelled “no PCR” in FIG. 7 show quantitative assessment of total cDNA yield applied to the samples set aside prior to PCR. The results labelled “6 PCR cycles” illustrate quantitative assessment of total cDNA yield of samples following 6 PCR cycles.

The results in FIG. 7 illustrate that the improved cDNA buffers described here provide significantly greater cDNA yield than do previously reported conditions.

vii) Cumulative Effect of Improved Conditions is to Significantly Improve PCR Yield as Compared to Previously Published Conditions.

To further assess the cumulative buffers changes and whether MMLV RTase and AMV RTase reverse transcriptases are effective at low input RNA levels the global amplification protocol (Protocol section (a) i, ii and iii) was applied to duplicate sets of a dilution of total mouse spleen RNA (Ambion) in which the input RNA ranges from 10 ng to 10 μg. For each reverse transcriptase reaction 85 u MMLV RTase plus 1 u AMV RTase reverse transcriptase was used. To assess PCR efficiencies 0.25 μL of the final PCR product was analysed by agarose gel electrophoresis.

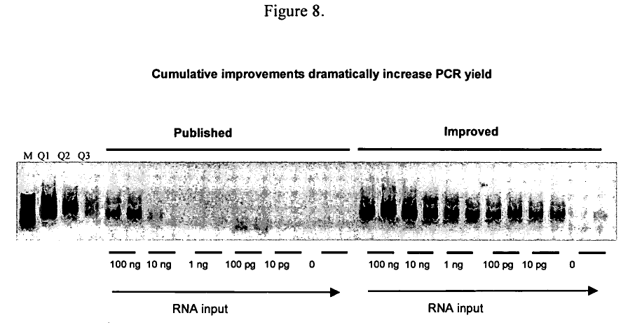

For all samples labelled “orig buffers” in FIG. 8 the buffers used were as outlined in the “Published” column of Table 1. For all samples labelled “new buffers” in FIG. 8 the buffers used were “RT Buffer 2+BSA” and “TdT buffer no DTT” as outlined in the “Improved” column of Table 1.

FIG. 8 shows agarose gel analysis, comparing global amplified cDNA yield achieved using previously published original conditions with that achieved using the improved conditions described herein.

Previously published global amplified cDNA preparation conditions yielded detectable PCR products for input RNA values of 100 ng (a product could very vaguely be detected at an input value of 10 ng). In contrast the improved conditions yielded substantial and detectable amounts of global amplified cDNA at input values as low as 10 μg RNA.

### REFERENCES

Brady, G., Barbara, M., and Iscove, N. N. (1990). Representative in vitro cDNA amplification from individual hemopoietic cells and colonies. Meth. Mol. Cell. Biol. 2, 17-25.
Brady, G., and Iscove, N. N. (1993). Construction of cDNA libraries from single cells. Methods Enzymol. 225, 611-623.
Chee, M., Yang, R., Hubbell, E., Berno, A., Huang, X. C., Stem, D., Winkler, J., Lockhart, D. J., Morris, M. S., and Fodor, S. P. (1996). Accessing genetic information with high-density DNA arrays. Science 274, 610-4.
Cumano, A., Paige, D. J., Iscove, N. N. and Brady, G. (1992) Bipotential precursors of B cells and macrophages in murine fetal liver. Nature, 356, 612-615.
Iyer, V. R., Eisen, M. B., Ross, D. T., Schuler, G., Moore, T., Lee, J. C., Trent, J. M., Staudt, L. M., Hudson, J., Jr., Boguski, M. S., Lashkari, D., Shalon, D., Botstein, D., and Brown, P. O. (1999). The transcriptional program in the response of human fibroblasts to serum. Science 283, 83-7
Kuribayashi-Ohta, K, Tamatsukuri S. Hikata M, Miyamoto C, and Furuichi Y “Application of oligo(dT)30-latex for rapid purification of poly(A)+ mRNA and for hybrid subtraction with the in situ reverse transcribed cDNA.”: Biochim Biophys Acta 1993, 1156: 204-12)
Liang, P., and Pardee, A. B. (1992). Differential display of eukaryotic messenger RNA by means of the polymerase chain reaction. Science 257, 967-71.
Lipshutz, R. J., Morris, D., Chee, M., Hubbell, E., Kozal, M. J., Shah, N., Shen, N., Yang, R., and Fodor, S. P. (1995). Using oligonucleotide probe arrays to access genetic diversity. Biotechniques 19, 442-7.
Lockhart, D. J., Dong, H., Byrne, M. C., Follettie, M. T., Gallo, M. V., Chee, M. S., Mittmann, M., Wang, C., Kobayashi, M., Horton, H., and Brown, E. L. (1996). Expression monitoring by hybridization to high-density oligonucleotide arrays. Nat Biotechnol 14, 1675-80.
Lopez-Fernandez, L A, and del Mazo J “Construction of subtractive cDNA libraries from limited amounts of mRNA and multiple cycles of subtraction.”: Biotechniques 1993, 15: 654-6, 658-9.
Maniatis, T., Fritsch, E. F., and Sambrook, J. (1982). Molecular Cloning: A Laboratory Manual (Cold Spring Harbor, N.Y.: Cold Spring Harbor University Press).
McClelland M, Hanish J, Nelson M, Patel Y. KGB: a single buffer for all restriction endonucleases. Nucleic Acids Res 1988, 11: 364.
Schena, M. (1996). Genome analysis with gene expression microarrays. Bioessays 18, 427-31.
Schena, M., Shalon, D., Davis, R. W., and Brown, P. O. (1995). Quantitative monitoring of gene expression patterns with a complementary DNA microarray. Science 270, 467-70.
Schraml, P, Shipman R, Stulz P, and Ludwig CU “cDNA subtraction library construction using a magnet-assisted subtraction technique (MAST).”: Trends Genet 1993, 9: 70-1.
Soares, M. B. (1997). Identification and cloning of differentially expressed genes. Curr Opin Biotechnol 8, 542-6.
Southern, E. M. (1996). DNA chips: analysing sequence by hybridization to oligonucleotides on a large scale. Trends Genet 12, 110-5.
Weaver, D L, Nufiez C, Brunet C, Bostock V, and Brady G. (1999). “Single-cell RT-PCR cDNA subtraction. In Molecular Embryology: Methods and Protocols.”, P. Sharpe and I. Mason, eds. (Totowa, N.J., USA: Humana Press), pp. 601-609).
Welcher, A A, Torres A R, and Ward D C “Selective enrichment of specific DNA, cDNA and RNA sequences using biotinylated probes, avidin and copper-chelate agarose.”: Nucleic Acids Res 1986, 14: 10027-44;

1-57. (canceled)

58. A collection of labelled target DNA molecules which are exonuclease derivatives of double-stranded DNA.

59. A collection according to claim 58, wherein the DNA molecules are fluorescently labelled.

60. A collection according to claim 58, wherein the DNA molecules are labelled by incorporation of labelled nucleotides

61. A collection according to claim 58, wherein the double-stranded DNA is cDNA.

62. A collection according to claim 61, wherein the cDNA is globally amplified cDNA.

63. A collection according to claim 61, wherein the labelled target DNA molecules are prepared from cDNA by a complexity reduction technique.

64. A collection according to claim 63, wherein the complexity reduction technique comprises a restriction digestion technique.

65. A collection according to claim 63, wherein the complexity reduction technique comprises a subtraction technique.

66. A collection according to claim 63, wherein the complexity reduction technique comprises a cDNA display technique.

67. A method of producing a collection of labelled target DNA molecules according to claim 58, comprising:
(i) subjecting double-stranded DNA molecules to exonuclease digestion to produce a collection of essentially single-stranded DNA molecules; and
(ii) labelling the single-stranded molecules.

68. A method according to claim 67, wherein the single-stranded molecules are labelled by the action of terminal transferase in the presence of labelled nucleotides.

69. A method of producing a collection of labelled target DNA molecules according to claim 58, comprising:
(i) treating double-stranded DNA to obtain a labelled double-stranded DNA population; and
(ii) effecting exonuclease digestion of the labelled population to produce a collection of essentially single-stranded labelled DNA molecules.

70. A method according to claim 69, wherein the labelled double-stranded DNA population is prepared by the action of terminal transferase in the presence of labelled nucleotides.

71. A method according to claim 69, wherein the labelled double-stranded DNA population is prepared by PCR in the presence of labelled nucleotides.

72. A method according to claim 67, wherein the exonuclease digestion comprises partial digestion of both strands of the double-stranded DNA molecules.

73. A method according to claim 67, wherein the exonuclease digestion comprises digestion of one strand of the double-stranded DNA molecules.

74. A method according to claim 73, wherein one strand of the double-stranded DNA molecules incorporates a restriction site, the molecules are treated with the appropriate restriction enzyme to produce double-stranded molecules having a sticky end, and the exonuclease digestion is effected from the blunt end of the double-stranded molecules.

75. A method according to claim 73, wherein the exonuclease digestion comprises digestion of the 3′-5′ strand of the double-stranded DNA molecules.

76. A method according to claim 67, wherein the exonuclease digestion is effected using exonuclease Ill.

77. A method according to claim 67, wherein the double-stranded DNA is cDNA, or a derivative thereof.

78. A method according to claim 77, wherein the cDNA is global amplified cDNA.

79. A method according to claim 78, wherein the global amplified cDNA is prepared by the steps of:
a) preparing a global cDNA population representative of gene expression in a biological sample of interest from mRNA of the sample by using primers and limiting concentrations of nucleotides;
b) homopolymer tailing the global cDNA population; and
c) amplifying the tailed global cDNA population.

80. A method according to claim 79, wherein at least one of steps a) and b) is effected in the presence of an acetate buffer.

81. A method according to claim 80, wherein the acetate buffer comprises Tris acetate incorporating potassium acetate and/or magnesium acetate.

82. A method according to claim 80, wherein step a) comprises reverse transcription of mRNA using primers capable of binding to the poly A tail of mRNA, the reverse transcription being performed in the presence of limiting concentrations of nucleotides.

83. A method according to claim 82, wherein the primers comprise a homopolymer T tract capable of binding to the poly A tail of mRNA.

84. A method according to claim 80, wherein step a) is effected in the presence of a buffer comprising:
20-500 mM Tris pH 8.3;
10-300 mM KCl;
1-20 mM MgCl₂;
2-200 mM Tris Acetate pH 7.9;
5-500 mM Potassium Acetate; and
1-10 mM Mg Acetate.

85. A method according to claim 79, wherein the reaction mixture of step a) further comprises:
5-500 μg/ml Glycogen;
0.01-5% NP-40;
0.02-10 u/ml RNase Inhibitor; and
70-80 μg/ml BSA.
2-200 μM dNTPs; and
0.01-100 μM oligonucleotide.

86. A method according to claim 85, wherein the reaction mixture comprises 0.1-5 μM oligonucleotide.

87. A method according to claim 80, wherein step b) comprises homopolymer tailing the global cDNA population, produced in step a), to produce a population of double-stranded DNA comprising both homopolymer A and homopolymer T tracts.

88. A method according to claim 87, wherein the homopolymer tailing is performed using terminal transferase.

89. A method according to claim 80, wherein step b) is effected in the presence of a buffer comprising:
10-250 mM Tris pH 8.3;
5-150 mM KCl;
0.5-10 mM MgCl₂;
2-200 mM Tris Acetate pH 7.9;
5-500 mM Potassium Acetate; and
1-10 mM Mg Acetate.

90. A method according to claim 79, wherein step b) is performed in the presence of bovine serum albumen.

91. A method according to claim 79, wherein step b) is performed in the absence of DTT.

92. A method according to claim 79, wherein the reaction mixture of step b) comprises:
2.5-250 μg/ml Glycogen
0.005-2.5% NP40;
0.1-10 mM COCl₂;
1-100 μM dNTPs;
0.005-2500 μM dT24;
0.01-5 u/ml RNase Inhibitor;
35-40 μg/ml BSA;
0.05-5 mM additional dATP; and
1-500 u/ml TdT enzyme.

93. A method according to claim 79, wherein step b) is performed in the presence of 0.5-2 mM CoCl₂.

94. A method according to claim 83, wherein step b) is performed in the presence of 1 mM CoCl₂.

95. A method according to claim 80, wherein step c) comprises amplifying the tailed double-stranded DNA by performing the polymerase chain reaction using the primers employed in step a).

96. A method according to claim 80, wherein step c) is effected in the presence of a buffer comprising:
20-500 mM Tris pH 8.3;
10-300 mM KCl;
1-20 mM MgCl₂;
2-200 mM Tris Acetate pH 7.9;
5-500 mM Potassium Acetate; and
1-10 mM Mg Acetate.

97. A method according to claim 79, wherein the reaction mixture of step c) comprises:
6-7 μM Oligonucleotide;
0.1-10 mM dNTPs;
2.5-250 μg/ml Glycogen;
0.03-3.3 mM COCl₂;
0.02-1% Triton X-100;
0.005-2.5% NP40;
35-40 μg/ml BSA;
0.05-5 mM additional dATP;
0.005 2500 μM dT24;
0.01-5 u/μl DNA Polymerase;
0.01-5 u/ml RNase Inhibitor; and
1-500 u/ml TdT enzyme.

98. A kit for the preparation of a collection of labelled target DNA molecules according to claim 58, the kit comprising:
(i) an exonuclease;
(ii) terminal transferase; and
(iii) labelled nucleotides.

99. A kit for the preparation of a collection of labelled target DNA molecules according to claim 58, the kit comprising:
(i) an exonuclease;
(ii) primers; and
(iii) labelled nucleotides.

100. A kit according to claim 99, further comprising reagents for PCR.

101. A kit according to claim 98, wherein the labelled nucleotides are fluorescently labelled.

102. A kit according to claim 98, wherein the exonuclease is exonuclease III.

103. A kit according to claim 98, further comprising reagents for production of cDNA.

104. A kit according to claim 103 comprising reagents for production of global amplified cDNA.

105. A kit according to claim 104, wherein the reagents for production of global amplified cDNA comprise acetate buffers.

106. A kit for carrying out the method of claim 84.

107. A kit according to claim 105, wherein the reagents for production of global amplified cDNA comprise:
Tris HCl;
KCl;
MgCl₂;
Tris acetate;
K acetate; and
Mg acetate.

108. A kit according to claim 107, including a first solution comprising:
20-500 mM Tris HCl;
10-300 mM KCl;
1-20 mM MgCl₂;
2-200 mM Tris acetate;
5-500 mM K acetate; and
1-10 mM Mg acetate.

109. A kit according to claim 107, including a second solution comprising:
10-250 mM Tris pH 8.3;
5-150 mM KCl;
0.5-10 mM MgCl₂;
2-200 mM Tris Acetate pH 7.9;
5-500 mM Potassium Acetate; and
1-10 mM Mg Acetate.

110. A kit according to claim 106, further comprising:
Glycogen;
COCl₂;
NP-40;
dNTPs;
dT;
RNase inhibitors;
bovine serum albumen;
dATP; and
Triton X-100.

111. A kit according to claim 110 comprising:
2.5-250 μM Glycogen;
0.1-10 mM COCl₂;
0.005-2.5% NP40;
1-100 μM dNTPs;
0.005-2500 μM dT;
0.01-5 u/ml RNase inhibitors;
70-80 μg/ml bovine serum albumen;
0.05-5 mM dATP; and
0.02-1% Triton X-100.

112. A kit according to claim 98, further comprising reverse transcriptase.

113. A kit according to claim 98, further comprising a DNA polymerase.

114. A kit according to claim 97, further comprising terminal transferase.
